(19) United States
(12) Patent Application Publication
(10) Pub. No.: US 2022/0313275 A1
(43) Pub. Date: Oct. 6, 2022

(54) RATCHET TOURNIQUET FOR CARDIOPULMONARY RESUSCITATION

(71) Applicant: 2607626 Ontario Limited, Sudbury (CA)

(72) Inventors: Samuel E. PETERS, Sudbury (CA); Marc TURGEON, Sudbury (CA)

(21) Appl. No.: 17/220,013

(22) Filed: Apr. 1, 2021

(51) Int. Cl. A61B 17/132

(52) U.S. Cl. CPC A61B 17/1327

(57) ABSTRACT

A tourniquet for cardiopulmonary resuscitation comprising a first strap having a first mating member and a second strap having a second mating member, wherein the first mating member engages the second mating member for securing the tourniquet around a thigh of a person on whom cardiopulmonary resuscitation is to be performed. The tourniquet further comprises a housing member interconnecting the first and second strap and a ratchet assembly for tightening the tourniquet. The ratchet assembly comprises a strap gear secured to the first strap, wherein the strap gear comprises a plurality of linearly arranged teeth; a ratchet comprising inwardly facing ratchet teeth; a pinion gear extending through the ratchet and engaging the strap gear, wherein the pinion gear includes a pair of pawls to engage the ratchet teeth; and a rotatable handle connected to the pinion gear to apply torque in order to tighten the tourniquet.

### TECHNICAL FIELD

The present invention relates generally to devices and methods for cardiopulmonary resuscitation and, more particularly, to tourniquets for cardiopulmonary resuscitation.

### BACKGROUND

Cardiac arrest is a leading cause of death in the United States, which according to the American Heart Association affects over a thousand people every day. Only one in ten people suffering from sudden cardiac arrest will survive, whereas it is estimated that at least three could survive if proper cardiopulmonary resuscitation (CPR) would be performed. Cardiac arrest occurs when the heart malfunctions, disrupting blood flow to the brain, lungs and other organs. The main purpose of CPR is to restore partial flow of oxygenated blood to the brain and heart in order to prevent permanent brain damage and/or death caused by the failure of other vital organs. Studies have demonstrated that myocardial blood flow during CPR is largely dependent on peripheral vascular resistance. Therefore, an important strategy for partially restoring blood flow to central organs is to restrict blood flow to the periphery.

The current ‘standard of care’ for cardiac arrest involves the administration of epinephrine (adrenaline), which is the most commonly used medication in cardiac arrest, and is the only drug recommended by the American Heart Association (AHA) for all arrests regardless of cardiac rhythm. Epinephrine is a vasoconstrictor that increases blood flow to central organs during CPR and its use in cardiac arrest originates from studies performed in dogs in the 1960's, which showed improve survival in dogs suffering from asphyxia. However, current studies have shown that epinephrine may actually worsen brain damage among cardiac arrest survivors. Alternative means of restricting peripheral blood flow during CPR should therefore be explored in order to limit permanent damage in patients having suffered cardiac arrest.

Tourniquets work by compressing muscle and other tissues surrounding arteries, thereby restricting or arresting blood flow distal to the tourniquet. Studies have demonstrated the benefits of combining chest compression with physical control of peripheral vascular resistance assisted by tourniquets. A new and improved tourniquet for cardiopulmonary resuscitation would thus be highly desirable.

### SUMMARY

The following presents a simplified summary of some aspects or embodiments of the invention in order to provide a basic understanding of the invention. This summary is not an extensive overview of the invention. It is not intended to identify key or critical elements of the invention or to delineate the scope of the invention. Its sole purpose is to present some embodiments of the invention in a simplified form as a prelude to the more detailed description that is presented later.

The present specification discloses a tourniquet for cardiopulmonary resuscitation that includes a first strap having a distal end and a proximal end, wherein the distal end comprises a first mating member, and a second strap having a distal end and a proximal end, wherein the distal end comprises a second mating member. The first mating member engages the second mating member for securing the tourniquet around a thigh of a person on whom cardiopulmonary resuscitation is to be performed.

The tourniquet further comprises a housing member interconnecting the first and second strap, wherein the proximal end of the first strap is secured inside the housing member.

The tourniquet comprises a ratchet assembly for tightening the tourniquet, the ratchet assembly comprising a strap gear secured to the first strap using a plurality of fasteners, wherein the strap gear comprises a plurality of linearly arranged teeth, a ratchet secured to the housing member, wherein the ratchet comprises inwardly facing ratchet teeth, and a pinion gear extending through the ratchet and engaging the strap gear. The pinion gear includes a pair of pawls to engage the inwardly facing ratchet teeth. The ratchet assembly further comprises a rotatable handle connected to the pinion gear to apply torque to the pinion gear for applying torque to the ratchet in order to tighten the tourniquet.

In another embodiment, the tourniquet comprises a spring plate located between the ratchet and the handle, wherein the spring plate comprises a plurality of springs that allow the pinion gear to move upward, disengaging the pinion gear from the strap gear.

In one embodiment, the rotatable handle comprises a plurality of finger grooves for gripping the handle when applying torque.

In one embodiment, the tourniquet further comprises a first clutch plate secured to the pinion gear and a second clutch plate secured to the pair of pawls. The clutch plates are sandwiched between the pinion gear and the pair of pawls for compressing the clutch plates together when force is applied to the springs via the rotatable handle. The force applied to the handle has to reach a desired threshold for the clutch plates to rotate when torque is applied, thereby allowing the pinion gear to engage the plurality of linearly arranged teeth of the strap gear. The induced tension on the strap is controlled by the friction between the clutch plates via the force from the springs.

In one embodiment, the tourniquet comprises a strap-holding member for securing the housing member to the second strap. The housing member comprises a first portion and a second portion, the first portion being secured to the first strap and the second portion being secured to the second strap via the strap-holding member.

In one embodiment, the tourniquet comprises a tightening member mechanically connected to the rotatable handle for further tightening the tourniquet.

In one embodiment, the tourniquet comprises a pressure monitor for monitoring an effective pressure range being applied.

In one embodiment, the first mating member of the first strap is a male end of a buckle and the second mating member of the second strap is a female end of a buckle.

The present specification also discloses a method of performing cardiopulmonary resuscitation that entails attaching a tourniquet to a thigh of a patient, tightening the tourniquet using a ratchet of the tourniquet, and performing cardiopulmonary resuscitation while the tourniquet is tightly attached to the thigh of the patient.

The present specification also discloses a method of applying a tourniquet to perform cardiopulmonary resuscitation that entails applying the tourniquet around a body part using a first and a second strap, securing the tourniquet by engaging a first mating member located on the first strap with a second mating member located on the second strap for securing the tourniquet around the body part, and tightening a ratchet assembly secured to the first strap for restricting blood flow to the body part.

In one embodiment, the ratchet assembly includes a strap gear comprising a plurality of linearly arranged teeth, a ratchet comprising inwardly facing ratchet teeth, and a pinion gear that includes a pair of pawls to engage the inwardly facing ratchet teeth.

In one embodiment, the step of tightening the ratchet assembly comprises applying force to a rotatable handle secured to the first strap to engage the plurality of linearly arranged teeth of the strap gear with the pinion gear of the ratchet for providing a compression force to the body part until blood flow to the body part has been restricted.

In one embodiment, applying torque engages the inwardly facing ratchet teeth with the pair of pawls of the pinion gear for preventing movement of the ratchet in an opposite direction to the applied torque. In addition, the springs provide the desired threshold for compressing two clutch plates and allow the pinion gear to engage the plurality of linearly arranged teeth of the strap gear.

Advantageously, the present invention allows for improved compression of a body part in order to better restrict blood flow to the body part. Since tourniquet-assisted CPR was shown to improve myocardial and cerebral perfusion, the present invention provides a new ratchet tourniquet designed to be applied to the thigh of a patient for restricting peripheral blood flow and maximize blood for vital tissues and the brain when performing CPR. The improved tightening mechanism provided by the ratchet tourniquet facilitates compression of the patient's thigh. The ratchet tourniquet of the present invention allows a medical professional to provide sufficient pressure for restricting peripheral blood flow and maintain blood within vital organs such as the heart, brain and lungs when a patient has suffered cardiac arrest. The ratchet provided with the tourniquet of the present invention allows the user to apply greater force to the body part and ensures that the tourniquet remains tightened by preventing movement of the ratchet in an opposite direction to the applied torque. The disclosed method of performing CPR using the ratchet tourniquet will increase chance of survival and/or minimize the risk of permanent brain damage associated with cardia arrest.

The ratchet tourniquet of the present invention can also be applied around an injured body part in order to restrict blood flow to the injured body part in order to stop bleeding and prevent hemorrhage. The improved tightening mechanism facilitates compression of the injured body part and provides sufficient pressure to stop arterial bleeding, which can save the injured person's life.

Other and further aspects and advantages of the present invention will be better understood upon the reading of the illustrative embodiments about to be described or will be indicated in the appended claims, and various advantages not referred to herein will occur to one skilled in the art upon employment of the invention in practice.

### BRIEF DESCRIPTION OF THE DRAWINGS

These and other features of the disclosure will become more apparent from the description in which reference is made to the following appended drawings.

FIG. 1 illustrates the ratchet tourniquet in accordance with an embodiment of the present invention.

FIG. 2 illustrates the ratchet tourniquet comprising two clutch plates in accordance with another embodiment of the present invention.

### DETAILED DESCRIPTION OF EMBODIMENTS

The following detailed description contains, for the purposes of explanation, numerous specific embodiments, implementations, examples and details in order to provide a thorough understanding of the invention. It is apparent, however, that the embodiments may be practiced without these specific details or with an equivalent arrangement. In other instances, some well-known structures and devices are shown in block diagram form in order to avoid unnecessarily obscuring the embodiments of the invention. The description should in no way be limited to the illustrative implementations, drawings, and techniques illustrated below, including the exemplary designs and implementations illustrated and described herein, but may be modified within the scope of the appended claims along with their full scope of equivalents.

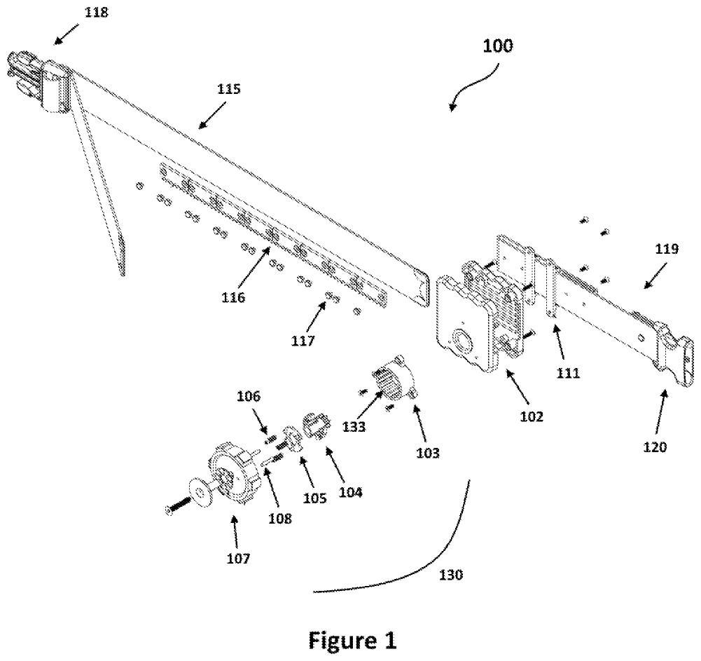

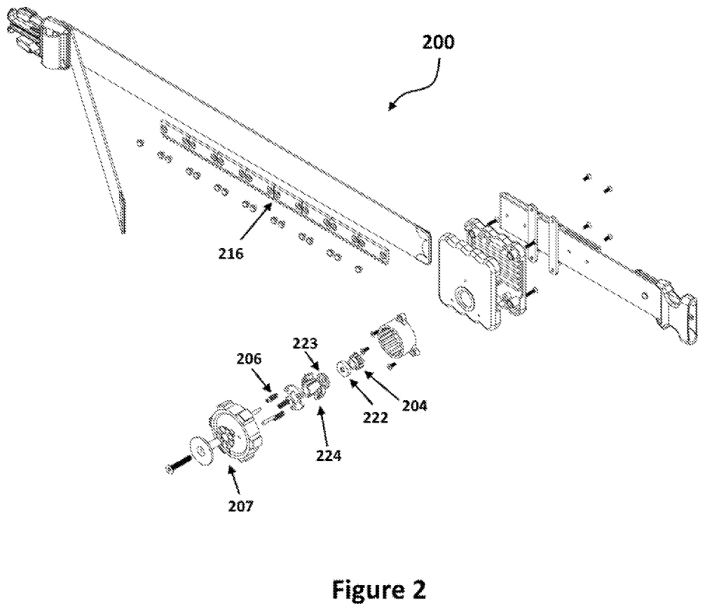

In accordance with embodiments of the present invention, a novel tourniquet for cardiopulmonary resuscitation is illustrated in FIGS. 1 and 2.

In the embodiment depicted in FIG. 1, the tourniquet 100 for cardiopulmonary resuscitation comprises a first strap 115 having a distal end and a proximal end, wherein the distal end comprises a first mating member 118, preferably a male end of a buckle. The tourniquet comprises a second strap 119 having a distal end and a proximal end wherein the distal end comprises a second mating member 120, preferably a female end of the buckle configured to connect to the male end. The tourniquet 100 is designed so that the first mating member 118 is able to engage the second mating member 120 in order to secure the tourniquet 100 around a thigh of a person on whom cardiopulmonary resuscitation (CPR) is to be performed. The tourniquet 100 further comprises a housing member 102 adapted for interconnecting the first and second straps 115, 119, wherein the proximal end of the first strap 115 is secured inside the housing member 102.

The tourniquet further comprises a ratchet assembly 130 for tightening the tourniquet around a body part of a patient upon whom CPR is to be performed. For example, the tourniquet may be tightened around a thigh of a patient. The tourniquet may be tightened around the thigh of the patient prior to performing cardiopulmonary resuscitation. When tightened around the thigh of the patient, the tourniquet increases vascular resistance to thereby improve the likelihood that CPR will be successful. The ratchet assembly 130 comprises a strap gear 116 secured to the first strap 115 using a plurality of fasteners 117. As depicted in FIG. 1, the strap gear 116 comprises a plurality of linearly arranged teeth. The ratchet assembly 130 includes a ratchet 103 secured to the housing member 102, e.g. by screws or other suitable fasteners. The ratchet 103 comprises inwardly facing ratchet teeth 133 to ensure unidirectional rotation of the ratchet 103. A pinion gear 104 extending through the ratchet 103 and engaging the strap gear 116 is also included in the ratchet assembly 130. The pinion gear 104 is provided with angled cuts to assist the pinion gear 104 to lift up and slide over the strap gear 116 when tension on the first strap 115 reaches a certain threshold. The pinion gear 104 includes a pair of pawls 124 that engage the inwardly facing ratchet teeth 133 to ensure unidirectional rotation of the ratchet 103. The ratchet assembly 130 further comprises a rotatable handle 107 connected to the pinion gear 104 in order to apply torque to the pinion gear 104 for applying torque to the ratchet 103 when tightening the tourniquet around a body part. In the embodiment depicted in FIG. 1, the rotatable handle 107 is fastened to the pinion by a screw 109. In order to maximize the torque applied to the ratchet by a user, the rotatable handle 107 may include a plurality of finger grooves 127 for gripping the handle 107 when applying torque. In order to loosen the strap in a slow and controlled manner, dowel pins 108 move the pawls 124 inward and away from the teeth on the ratchet 103, when the handle 107 is turned in a counter clockwise direction, thereby preventing a rush of blood flow back into the limb.

In the embodiment depicted in FIG. 1, the tourniquet further comprises a spring plate 105 located between the ratchet 103 and the handle 107. The tourniquet may also include a plurality of springs 106 between the handle 107 and the spring plate 105. The force applied to the handle 107 is transferred to the pinion gear 104 via the springs 106, or spring force. More specifically, the spring plate 105 locates the springs 106 and distributes the spring force over the pinion gear 104. The spring force pushes down on the pinion gear 104 until the strap tension reaches a certain threshold, and then allows the pinion gear 104 to move along the strap gear 116. The point at which the pinion gear moves along the strap gear 116 corresponds to the maximum tension that can be applied on the first strap 115. Adjusting the maximum tension can easily be accomplished by replacing the springs 106 with different load capacity springs since the maximum tension is controlled by the springs 106. Consequently, the maximum pressure applied to the limb is controlled by the springs 106. Moreover, when the force on the handle 107 is released, the pinion gear remains engaged with the ratchet due to the pawls interlocking with the teeth of the ratchet 103. Two dowel pins 108 may be provided to fit into slots within the pawls.

In the embodiment depicted in FIG. 1, the housing member 102 comprises a first portion and a second portion, the first portion being secured to the first strap 115 and the second portion being secured to the second strap 119 via a strap-holding member 111. In operation, the user wraps the first and second straps 115, 119 around the thigh of the patient and connects the male and female ends 118, 120 of the buckle. Pulling on the free end 119 of the first strap 115 ensures that the first strap 115 is under tension before the tourniquet is tightened by rotating the handle 107 which causes the pinion gear 104 to rotate within the ratchet 103. The pawls of the pinion gear 104 incrementally engage the internal teeth of the ratchet 103 to prevent counter-rotation. The pinion gear 104 engages the teeth of the strap gear 116, causing the strap gear 116 to move linearly (like a rack relative to a pinion), thereby causing the first strap 115 to move, thus tightening the tourniquet.

In the embodiment depicted in FIG. 2, the tourniquet 200 for cardiopulmonary resuscitation further comprises a first clutch plate 222 secured to the pinion gear 204 and a second clutch plate 223 secured to the pair of pawls 224. The clutch plates are sandwiched between the pinion gear and the pair of pawls such that when force is applied to the springs 206 via the handle 207, the clutch plates 222,223 are compressed together. The clutch plates 222,223 ensure that sufficient force is applied to the handle 207 in order to reach a desired threshold for the clutch plates 222,223 to rotate when torque is applied, which allows the pinion gear 204 to engage the plurality of linearly arranged teeth of the strap gear 216 to thereby displace the strap gear 216 and thus tighten the tourniquet. The induced tension on the first strap 215 is controlled by the friction between the clutch plates 222, 223 via the force from the springs 206.

In another embodiment, the tourniquet further comprises a tightening member connected to the rotatable handle such as a shaft mechanically connected to the rotatable handle for further tightening the tourniquet. The person skilled in the art will readily acknowledge that other variations of a shaft may be used as a tightening member without deviating from the teachings of the present invention. For example, the shaft may be replaced with a V-shape, T-shape or any other suitable shape for maximizing torque applied to the pinion gear and ratchet in order to further tighten the tourniquet.

In another embodiment, the tourniquet further comprises a pressure monitor for monitoring pressure being applied in order to ensure an effective pressure range, for example, between 100 mm Hg to 250 mm Hg for a patient's thigh. An optimal pressure range may then be determined by a medical professional depending on the body part on which the tourniquet is being applied, a patient's BMI, the nature of an injury, the extent of bleeding as well as other parameters well known in the art. The pressure monitor may comprise a strong air bladder with a tube attached to a sphygmomanometer. Other variations of a pressure monitor may include a pressure plate located within the housing member to measure the pressure being applied. The pressure monitor would allow the medical professional or any other user of the tourniquet to prevent undue harm to the limb for which it is being applied on, and also ensure that the target pressure is being reached. As such, an optimal pressure range can easily be reached.

Another aspect of the invention is a novel method of performing cardiopulmonary resuscitation using a tourniquet. The method comprises attaching a tourniquet to a thigh of a patient, tightening the tourniquet using a ratchet of the tourniquet, and performing cardiopulmonary resuscitation while the tourniquet is tightly attached to the thigh of the patient.

Another aspect of the invention is a novel method of applying a tourniquet to perform cardiopulmonary resuscitation. The method comprises applying the tourniquet around a body part using a first and a second strap, securing the tourniquet by engaging a first mating member located on the first strap with a second mating member located on the second strap for securing the tourniquet around the body part, and tightening a ratchet assembly secured to the first strap for restricting blood flow to the body part. The ratchet assembly comprises a strap gear comprising a plurality of linearly arranged teeth, a ratchet comprising inwardly facing ratchet teeth, and a pinion gear comprising a pair of pawls to engage the inwardly facing ratchet teeth.

When tightening the ratchet assembly, force is first applied to a rotatable handle secured to the first strap. Torque is then applied to the rotatable handle in order to engage the plurality of linearly arranged teeth of the strap gear with the pinion gear of the ratchet for providing a compression force to body part until blood flow to the body part has been restricted. The torque applied to the rotatable handle engages the inwardly facing ratchet teeth with the pair of pawls of the pinion gear for preventing movement of the ratchet in an opposite direction to the applied torque. In addition, the force applied by the springs has to reach a desired threshold for compressing the two clutch plates sandwiched between the pinion gear and the pair of pawls to allow the pinion gear to engage the plurality of linearly arranged teeth of the strap gear.

In an alternative embodiment, the ratchet tourniquet may be applied and sufficiently tightened around an injured body part in order to restrict blood flow to the injured body part, thereby minimizing the risks of hemorrhage.

In another embodiment, the ratchet tourniquet may be applied and sufficiently tightened around an injured body part by the injured person in order to restrict blood flow to the injured body part.

It is to be understood that the singular forms “a”, “an” and “the” include plural referents unless the context clearly dictates otherwise. Thus, for example, reference to “a device” includes reference to one or more of such devices, i.e. that there is at least one device. The terms “comprising”, “having”, “including”, “entailing” and “containing”, or verb tense variants thereof, are to be construed as open-ended terms (i.e., meaning “including, but not limited to,”) unless otherwise noted. All methods described herein can be performed in any suitable order unless otherwise indicated herein or otherwise clearly contradicted by context. The use of examples or exemplary language (e.g. “such as”) is intended merely to better illustrate or describe embodiments of the invention and is not intended to limit the scope of the invention unless otherwise claimed.

While several embodiments have been provided in the present disclosure, it should be understood that the disclosed systems and methods might be embodied in many other specific forms without departing from the scope of the present disclosure. The present examples are to be considered as illustrative and not restrictive, and the intention is not to be limited to the details given herein. For example, the various elements or components may be combined or integrated in another system or certain features may be omitted, or not implemented.

In addition, techniques, systems, subsystems, and methods described and illustrated in the various embodiments as discrete or separate may be combined or integrated with other systems, modules, techniques, or methods without departing from the scope of the present disclosure. Other items shown or discussed as coupled or directly coupled or communicating with each other may be indirectly coupled or communicating through some interface, device, or intermediate component whether electrically, mechanically, or otherwise. Other examples of changes, substitutions, and alterations are ascertainable by one skilled in the art and could be made without departing from the inventive concept(s) disclosed herein.

1. A tourniquet for cardiopulmonary resuscitation, the tourniquet comprising:
a first strap having a distal end and a proximal end, wherein the distal end comprises a first mating member;
a second strap having a distal end and a proximal end, wherein the distal end comprises a second mating member;
wherein the first mating member engages the second mating member for securing the tourniquet around a thigh of a person on whom cardiopulmonary resuscitation is to be performed;
a housing member interconnecting the first and second strap, wherein the proximal end of the first strap is secured inside the housing member; and
a ratchet assembly for tightening the tourniquet, the ratchet assembly comprising:
a strap gear secured to the first strap, wherein the strap gear comprises a plurality of linearly arranged teeth;
a ratchet secured to the housing member, the ratchet comprising inwardly facing ratchet teeth;
a pinion gear extending through the ratchet and engaging the strap gear, wherein the pinion gear includes a pair of pawls to engage the inwardly facing ratchet teeth; and
a rotatable handle connected to the pinion gear to apply torque to the pinion gear for applying torque to the ratchet in order to tighten the tourniquet.

2. The tourniquet of claim 1, further comprising a spring plate located between the ratchet and the handle, wherein the spring plate comprises a plurality of springs for disengaging the handle when not in use.

3. The tourniquet of claim 1, wherein the rotatable handle comprises a plurality of finger grooves for gripping the handle when applying torque.

4. The tourniquet of claim 2, further comprising:
a first clutch plate secured to the pinion gear, and
a second clutch plate secured to the pair of pawls, wherein the clutch plates are sandwiched between the pinion gear and the pair of pawls for compressing the clutch plates together when force is applied to the springs via the handle.

5. The tourniquet of claim 4, wherein the force applied to the handle has to reach a desired threshold for the clutch plates to rotate when torque is applied, thereby allowing the pinion gear to engage the plurality of linearly arranged teeth of the strap gear.

6. The tourniquet of claim 1, further comprising a strap-holding member for securing the housing member to the second strap.

7. The tourniquet of claim 6, wherein the housing member comprises a first portion and a second portion, the first portion being secured to the first strap and the second portion being secured to the second strap via the strap-holding member.

8. The tourniquet of claim 1, further comprising a tightening member mechanically connected to the rotatable handle for further tightening the tourniquet.

9. The tourniquet of claim 1, further comprising a pressure monitor for monitoring an effective pressure range being applied.

10. The tourniquet of claim 1, wherein the strap gear is secured to the first strap using a plurality of fasteners.

11. The tourniquet of claim 1, wherein the first mating member of the first strap is a male end of a buckle and the second mating member of the second strap is a female end of a buckle.

12. A method of performing cardiopulmonary resuscitation, the method comprising:
attaching a tourniquet to a thigh of a patient,
tightening the tourniquet using a ratchet of the tourniquet, and
performing cardiopulmonary resuscitation while the tourniquet is tightly attached to the thigh of the patient.

13. A method of applying a tourniquet, the method comprising:
applying the tourniquet around a body part using a first and a second strap;
securing the tourniquet by engaging a first mating member located on the first strap with a second mating member located on the second strap for securing the tourniquet around the body part; and
tightening a ratchet assembly secured to the first strap for restricting blood flow to the body part,
wherein the ratchet assembly comprises a strap gear comprising a plurality of linearly arranged teeth, a ratchet comprising inwardly facing ratchet teeth, and a pinion gear comprising a pair of pawls to engage the inwardly facing ratchet teeth.

14. The method of claim 13, wherein tightening the ratchet assembly comprises:
applying force to a rotatable handle secured to the first strap in order to engage a plurality of springs, and
applying torque to the handle in order to engage the plurality of linearly arranged teeth of the strap gear with the pinion gear of the ratchet for providing a compression force to an injured body part until blood flow to the body part has been restricted.

15. The method of claim 14, wherein applying torque engages the inwardly facing ratchet teeth with the pair of pawls of the pinion gear for preventing movement of the ratchet in an opposite direction to the applied torque.

16. The method of claim 14, wherein the force applied to the handle has to reach a desired threshold for compressing two clutch plates sandwiched between the pinion gear and the pair of pawls to allow the pinion gear to engage the plurality of linearly arranged teeth of the strap gear.

17. A tourniquet for restricting blood flow to an injured body part, the tourniquet comprising:
a first strap having a distal end and a proximal end, wherein the distal end comprises a first mating member;
a second strap having a distal end and a proximal end, wherein the distal end comprises a second mating member;
wherein the first mating member engages the second mating member for securing the tourniquet around an injured body part;
a housing member interconnecting the first and second strap, wherein the proximal end of the first strap is secured inside the housing member; and
a ratchet assembly for tightening the tourniquet, the ratchet assembly comprising:
a strap gear secured to the first strap, wherein the strap gear comprises a plurality of linearly arranged teeth;
a ratchet secured to the housing member, the ratchet comprising inwardly facing ratchet teeth;
a pinion gear extending through the ratchet and engaging the strap gear, wherein the pinion gear includes a pair of pawls to engage the inwardly facing ratchet teeth; and
a rotatable handle connected to the pinion gear to apply torque to the pinion gear for applying torque to the ratchet in order to tighten the tourniquet.
